(19) United States
(12) Patent Application Publication
(10) Pub. No.: US 2025/0057406 A1
(43) Pub. Date: Feb. 20, 2025

(54) SYSTEMS AND METHODS FOR DRYING ENDOSCOPIC DEVICES

(71) Applicant: GI SCIENTIFIC, LLC, Arlington, VA (US)

(72) Inventors: Scott MILLER, Arlington, VA (US); Frank CARTER, Wormleysburg, PA (US); Carl GAUGER, Kansas City, MO (US)

(21) Appl. No.: 18/938,650

(22) Filed: Nov. 6, 2024

Related U.S. Application Data
(63) Continuation of application No. PCT/US2023/021120, filed on May 5, 2023.
(60) Provisional application No. 63/339,497, filed on May 8, 2022.

(51) Int. Cl. A61B 1/12

(52) U.S. Cl. CPC A61B 1/122

(57) ABSTRACT

Devices, methods and kits for cleaning and/or drying endoscopic instruments, such as endoscopes, are provided. In one aspect, a drying device for use with an endoscopic instrument comprises an elongate member configured for advancement through an internal lumen within the endoscopic instrument and a drying member removably coupled to a portion of the elongate member. At least a portion of the drying member has an outer diameter greater than an outer diameter of the elongate member and may be shaped and configured to substantially maintain contact with a wall of the lumen as the elongate member is advanced through the lumen. The elongate member and the drying member are sterilized or disinfected so that the disinfected and/or sterilized device channel is not compromised by the drying process.

### BACKGROUND

Endoscopes are used and reprocessed numerous times each day to deliver highly advanced optical performance, consistent real-time imaging transmission, predictable scope handling and other functionality important to successful diagnosis and treatment of clinical conditions. This also occurs in non-medical applications involving the inspection, cleaning and repair of remote locations with non-medical endoscopes. This includes, by way of example, but not limitation, the inspection and repair of hydraulic lines, oil field pipelines, oil refinery lines and lumens, sewer and plumbing lines, the internal areas of a combustion engine and other non-medical applications involving remote visualization of an area that benefits from remote access and assessment.

Endoscopes are high technology instruments, typically having advanced, expensive optical chips at the distal end of the scope to facilitate exceptional visualization. These imaging signals are captured on the chip and communicated in turn through high definition image transfer technology involving sophisticated software and imaging processing hardware that processes the optical signals. These signals in turn are translated and projected through the software and processor at numerous frames per second to an imaging screen, console or other means of transmitting the image to a user distant from the optical chip.

The exceptional imaging capability of endoscopes has enabled numerous advances in medical and non-medical fields. This is due in significant part to the combination of excellent optical performance and scope handling joined to the reusable nature of nearly all endoscopes. This powerful combination allows for advanced, premium optical elements to be made available at a reasonable per use cost due to the ability to clean, disinfect (as applicable) and reuse the endoscope with its advanced optical capability. The ability to reuse these scopes effectively spreads the high cost of the endoscope's capability across multiple procedures/uses, thereby enabling reasonable, low cost access to advanced technologies for multiple beneficial uses on a global basis. Endoscopes with these advanced optical capabilities are too expensive to be used once and discarded. In addition, the environmental impact of discarding the advanced electronics that facilitate the endoscope's capability is considerable, unwarranted and unsafe for the environment. Reusable scopes provide a way to make peak optical capability available for a variety of procedures where otherwise one would not be able to afford the cost to use such technology.

Even with the considerable advances and capabilities offered by reusable endoscopes, recent concerns have arisen regarding one's ability to consistently and predictably clean and thereby remove all soil and biomatter that contaminates endoscopes during use. Successful cleaning is the critical step to support disinfection and/or sterilization (as applicable) to reprocess these scopes for their next use. Cleaning non-medical scopes is also important to avoid inhibiting scope performance with the next use because of retained matter that can accumulate and adversely impact scope performance. This applies to both non-robotic scopes and scopes connected to or otherwise used with robotic technology to use remote visualization to see, navigate and treat, as applicable.

Multiple contamination-related reprocessing issues leading to potential patient infections and/or scope performance issues have been noted with these scopes. These include issues with the cleanliness of reusable valves used to facilitate suction and air/water expression, the presence of residual matter that cannot be consistently removed from the complex distal end of certain scopes (especially duodenoscopes and endoscopic ultrasound scopes), and concerns regarding successful cleaning of the long biopsy/working channel(s) in certain scopes that are important for passing instruments to the distal end of the scope.

Nearly all of these issues are addressable through the use of new, relatively low cost technologies and practices that have been created in response to these concerns and which can be applied in the context of current workflows and procedure economics, and which are environmentally friendly, especially when compared to single-use scope alternatives. These relatively low cost technologies and practices include the use of single-use disposable tubing and disposable valves instead of reusable tubing and valves, the use of sterile, single-use endoscopic shields to seal the complex distal end of the scope during use and initial pre-cleaning instead of leaving this area open and exposed to contamination, the use of forced-air drying, improved adherence to reprocessing approaches, and the implementation of post-procedure culturing and monitoring to address other areas of concern.

With all of these advances, an area that remains to be addressed is the cleaning of the internal lumens of the endoscope. During a medical procedure, the internal biopsy and suction channels become heavily contaminated with bacteria, biomatter and debris through the passing of multiple instruments through the biopsy channel and through the actuation of suction to remove mucus, debris and other matter that may obscure the physician's visualization during the procedure. All of these activities benefit patients by delivering care through the scope in a less invasive manner, but in parallel with these beneficial activities the scope experiences heavy contamination of these channels, which then must be cleaned effectively to return the scope to use for the next patient (or non-medical use) without exposing the next patient (or non-medical use) to the risk of a scope-related infection (or a poor performing, unclean non-medical scope). It is well known that without successful cleaning, an endoscope cannot be disinfected or sterilized successfully. Unremoved biomatter and debris act as a shield for pathogens, protecting the pathogens from being killed by disinfectants and sterilants used to reprocess the scope. Additionally, unremoved biomatter, and pathogens create the opportunity for organisms to attach to surfaces inside the scope, engage in replication and form biofilm, which makes removal of these organisms particularly difficult and which, in turn, creates risk for the transmission of multi-drug resistant infections through the scope. Biofilm can replicate, detach and then attach in a new location and repeat this process, while also recruiting other organisms into the biofilm during the process, creating additional multi-drug resistant organisms (MDRO) that cannot be effectively treated with antibiotics. MDRO infections are exceptionally dangerous and have resulted in multiple deaths around the world from contaminated endoscopes that were not reprocessed successfully.

Another significant unmet need with these instruments and with this process is a downstream issue associated with drying the instrument after the cleaning and/or disinfection or sterilization process is completed. Most, if not all, of the disinfection or sterilization approaches involve some element of liquid or gas delivery of the disinfectant or sterilant, followed in most protocols by flushing with an agent, such as water or sterile water to rinse and aid in removal and/or or in select and very limited instances evaporation of the agents used to disinfect or sterilize.

In all of these processes, a critical final step after the disinfection or sterilization is drying of the instrument. For external surfaces of the medical device, this aspect of the process is fairly straight-forward, but for recessed areas and especially relatively narrow and sometimes long channels, the drying of these areas is difficult, lengthy and not predictable or consistent. Failing to effectively dry counteracts the benefits of the entire reprocessing approach, including the disinfection and/or sterilization steps. This is because wet surfaces promote the proliferation of pathogens and biofilm. If the instrument is dry, then pathogens and biofilm cannot grow. Hydration is a central element for the growth of pathogens and biofilm, which lead to infections, including nosocomial, drug-resistant and other deadly infections. For non-medical uses, residual hydration promotes the growth of biofilm and pathogens which can effect device performance and even expose a user eventually to one or more pathogens.

Current approaches to drying have applied a number of inconsistent, variable methods that do not provide consistent assurance that the moisture within these channels and recessed areas is removed and therefore the remote and/or recessed areas are dry. Unsuccessful attempts have been made to just hang devices vertically, so wet channels are positioned so the effect of gravity causes water inside channels to eventually run down and evaporate. This approach has been disproven with evidence that even after days in this position, these channels are not consistently and completely dry.

Attempts to overcome these issues have included flushing these channels with alcohol, under the premise this may accelerate drying and act as an additional bacteriostatic agent (though the instrument in medical applications had already been disinfected or sterilized at this point). Data shows this does not clearly accelerate drying times or drying effectiveness and can even lengthen the drying process in certain instants. In addition, there are concerns that the use of alcohol may actually fixate certain otherwise inert or innocuous bacteria and pathogens in the channel that in turn can be passed on to the patient or user.

Other approaches involve using air drying approaches, which is inherently difficult, time-consuming and variable due to the recessed and at times long thin channels that must be dried. Certain of these channels are four to six feet long with diameters that are only a few millimeters or less. Efforts to dry include aiming compressed air at or into a channel, or hanging the instrument in a cabinet with circulating air that uses motorized fans to blow air into small spaces to improve drying. Certain of these approaches have filtered air that is blown by a motorized fan through tubing into tight areas, such as endoscope channels and labeled “forced air drying.” Even these more proactive and more targeted approaches have variable results, which is due, at least in part, to the distance that the air must travel to dry the channel, the variable channel diameters of the channels, which impact the air velocity, and the surface tension of the water, which can be greater than the force of the air at different points, preventing effective and timely removal of the water. Additionally, the tight spaces essentially trap or help retain and promote a humid environment, inhibiting evaporation. Challenges beyond even these significant difficulties include endoscopes and other instruments with curved internal channels and lumens where one or more channels come together creating even more variable surfaces, such as Y-junctions and connectors where water can pool and areas adjacent to and/or outside of valves that interact with a channel.

Accordingly, it would be desirable to provide improved systems and methods for drying endoscopic instruments, such as endoscopes, and other instruments with remote channels and lumens, after they have been cleaned so that the channels and lumens can be effectively sterilized or disinfected. In particular, it would be desirable to provide devices that can effectively remove all fluid and moisture from the internal lumens of endoscopic technologies, including fluid residing in crevasses, scratches, Y junctions, or other irregularities.

### SUMMARY

Devices and methods are provided for drying endoscopic instruments, such as endoscopes, after they have been cleaned. These devices and methods are particularly useful for rapidly and consistently drying the internal lumens or other spaces within the endoscopic instruments. Devices, kits and methods are also provided for both cleaning and drying endoscopic instruments, and for testing to confirm successful cleaning and drying. The disclosed innovations address the multiple limitations with current approaches for cleaning and/or drying endoscope lumens or channels, and provide new, important capabilities to improve cleaning and drying performance

The methods and devices disclosed herein may be used with, or may be incorporated into, a variety of different reusable or disposable endoscopic instruments and devices that include internal lumens or other internal spaces, such as endoscopes, trocars, cannulas, dilatation devices, Foley catheters and other in-dwelling catheters, guidewires, central venous catheters, bipolar or monopolar electrosurgical or ultrasonic devices, arterial lines, drainage catheters, peripherally inserted central catheters, endotracheal tubes, feeding tubes, ventilators, respirators, robotic surgery devices and other devices that in-dwell, penetrate and/or navigate in the body. The dimensions of the drying devices disclosed herein would, or course, be adjusted for the size of the particular instrument or device.

In one embodiment, a drying device for use with an endoscopic instrument comprises an elongate member configured for advancement through a lumen within the endoscopic instrument and a drying element coupled to at least a portion of the elongate member. The drying element includes certain portions that have an outer dimension (e.g., diameter, width, height or other dimension) greater than the outer dimension of the elongate member.

In certain embodiments, the drying device is sterilized or disinfected so that the disinfected and/or sterilized device channel is not compromised by the drying process. Instead, when the drying device is passed through the already disinfected or sterilized device channel, water and moisture is removed without altering or contaminating the disinfected or sterilized state of the device with the channel.

In certain embodiments, the drying device includes more than one drying member. For example, in one such embodiment, the drying device includes 2-10 drying members, preferably 2-6 drying members. The drying members may be coupled to each other to provide a string of such drying members along the elongate element, or separated by various distances, to increase the effectiveness of the device.

The drying members may be discs, fins, squeegees, or other projections extending outward from the elongate member. In embodiments, the drying members are shaped to create consistent circumferential contact with the interior wall of an endoscope channel, such as a biopsy or suction channel, as the elongate member is advanced therethrough.

In certain embodiments, the drying members preferably have a substantially annular, circumferential, cylindrical or conical shape with at least one portion of the element having a diameter approximately equal to or slightly larger than the diameter of the internal lumen. In an exemplary embodiment, the diameter of the proximal and distal end portions is about 1 to about 1.5 times the diameter of the internal lumen, preferably about 1 to about 1.25 times this diameter. This avoids deflection of the proximal and distal end portions, thereby reducing the buckling and the creation of a gap between the drying element and the internal wall of the lumen.

In certain embodiments, the elongate member of the drying device comprises a filament, wire, tube, shaft or other element capable of advancement into an internal channel, including a long, curved, or other channel that is difficult to access because it is recessed or internal. In embodiments, this element can be grasped at an end once it is pulled through the internal channel and then pulled through the channel as part of the drying process. It also can be advanced from the other end through pushing the advancement element. This element may include a version that is able to advance air through the device and into the channel, including to a removed location well inside the channel and beyond the typical effectiveness of forced air drying in a channel.

The drying member may comprise one or more substantially annular or cylindrical members spaced from each other along the elongate member. The cylindrical members are configured to maintain contact with the channel wall to displace and remove any moisture as the elongate element travels through the channel. The elongate element may be advanced through being pushed, pulled or by other means, including back and forth agitation, advancement and withdrawal based on a pattern. The elongate element may travel through the scope by connection to an automated or mechanized approach or any other means of moving the channel drying element through the channel to displace, absorb and or remove water at least in part through direct contact with the surface area to be dried.

In embodiments, the drying element of the invention may be any shape or form that can displace water and moisture through advancement and withdrawal, including a squeegee, disc, fin, cylinder, series of absorptive strips or other absorptive material or matter including a sponge, venturi or inverted venturi, oblong and round shapes, a ball or spherical shaped drying element.

In embodiments, the drying member may comprise a series of one or more cylindrical squeegees that are designed to make wall contact with the internal walls of the channel to remove fluid and moisture from the channel. These cylindrical squeegees may be arranged in any manner that is effective at removing fluid and moisture from the channel. This includes in groups of two and then three squeegees, or vice versa, or in other configurations of squeegees, such as groups of one and then two squeegees, two and then four, three and three, four in a row, five in a row or any other number and configuration of squeegees that is effective for the removal of water and moisture, depending on the channel diameter, length, curves, junctures with other channels and other factors.

In embodiments, certain of these squeegees may be of different heights in order to make the device more adaptable to use in channels of different sizes. For example, the device may have a squeegee that is 5 to 5.2 mm in diameter followed by a smaller squeegee that is between 2 to 2.2 mm in diameter, followed by another squeegee that is 3.8 to 4 mm in diameter to create variable wall contact across a variety of channel sizes. In embodiments, the height and arrangement of squeegees is not limited to this description and these diameters, placements, squeegee thickness and other shall vary based on what is most effective for the drying for a given channel drying application.

The spacing between squeegees and any other elements, including drying elements, may be any spacing that supports rapid and predictable channel drying, including a spacing of 1 mm, 1.25 mm, 1.5 mm, 2 mm, close spacing of 1.25 mm and then a 4 mm gap and then spacing of 1.25 to 1.5 mm or other spacing closer or farther or more variable that optimizes and supports rapid and predictable channel drying. Spacing, diameters and alignment and grouping of drying elements may vary based on channel size, channel material, the number of curves to navigate around, the number of junctions with other channels, if any, the channel dryer material, and other factors specific to the given channel drying application, in embodiments.

The thickness of squeegees and fins are any thickness that aids in channel drying and the positioning of the fins and squeegees to dry and, in embodiments, clean. In embodiments, this includes squeegee and fin thickness of between 0.2 and 0.5 mm, other variants with a thickness of 0.5 to 1.0 mm; other variances with tapered thickness with the thickness greater at the base of the squeegee or fin and tapering closer to the channel wall to enhance deflection and conformance of the element to the channel wall, while providing support and a certain rigidity and flexibility around the advancing element.

The annular members are spaced from each other along the elongate member. In some embodiments, the distance between each of the annular members is substantially equal. In other embodiments, this distance may vary between some or all of the annular members. For example, the device may include first, second and third annular members, with the first and second annular members being spaced from each other by a distance greater than the second and third annular members.

In one embodiment, the device includes at least first and second groups of annular members with each group having a plurality of annular members. The annular members within each group may be spaced substantially equally from each other, or they may vary in spacing. The first group is spaced from the second group by a distance greater than the spacing within each of the first and second groups.

The fins, discs, squeegees or other annular member, or other drying member, may be at any angle to the channel wall that promotes and enhances drying, including perpendicular, angled backward and an angle of between 1 and 89 degrees, angled forward at an angle of between 1 and 89 degrees or any other angle that promotes drying. In embodiments, these angles shall not be limited to this description and shall vary based on what is most effective for the drying for a given channel drying application.

In another embodiment, elongate member comprise a rod member having a first end, a second end and loop coupled to the first end. The device may further include a swab member having a first end attached to the loop and a second end comprising a tail. The swab member may have first and second ends where the first end is attached to the loop and the second end comprises a tail such as the tail of a kite where the tail can be of any length. The swab member may comprise one or more materials that can be braided or twisted with the tail end of the swab member comprising the unbraided or untwisted ends of the material or materials that comprise the swab member. In addition, the swab member may comprise a foam material and/or a microfiber material.

In addition, the rod member may comprise one continuous polymer rod with the looped end of the rod member comprising one thickness or diameter and the straight end of the rod member comprising two thicknesses or two diameters of the looped end of the rod. The two thicknesses or diameters of the polymer rod that form the straight end of the rod member may be fused or bonded together, laminated together, or co-extruded.

In embodiments, the drying device includes a centering element, so that the drying element(s) remain centered as they are advanced through the channel to dry. This centering element prevents a drying element from losing contact with the wall of the channel being dried, enhancing drying action. The centering element may be any shape that keeps the device generally centered in the lumen and prevents this deflection, with a preferred embodiment being a cylindrical shape with a tapered distal end. When this sort of misalignment occurs, which is an issue with existing brushes and pull thru devices, the brushes and other elements are pulled to one side of the lumen as the devices are pulled around curves, corners and junctions of lumens, with the result being contact with the lumen wall and the drying element (whether a brush, pull thru or other device) is minimized, altered in an adverse way, or lost, resulting in an adverse impact on the effectiveness of the drying approach. By placing a centering element at the front or back, or both of the device, this issue is corrected, resulting in more consistent, effective drying, especially around curves, corners, channel junctions and other complex areas inside an endoscope or other endoscopic instrument or device.

In a preferred embodiment, the centering element is between 50 percent and 90 percent of the diameter of the lumen being cleaned, with a further preferred embodiment having a diameter or height between 70 percent and 85 percent of the diameter of the lumen being cleaned.

In some embodiments, the centering element may comprise a series of shaped elements projecting from the elongate member or the navigation member. This series of shaped elements may, for example, comprise struts, spikes, or other projections extending radially outward from the elongate member and sized to substantially center the elongate member and/or the drying/cleaning elements within the internal channel of the endoscopic device.

In embodiments, the elongate member comprises an attachable pushing and pulling element, such as a navigation element, that can be advanced from the proximal end of an internal lumen of an endoscopic instrument, such as a biopsy channel on an endoscope, to the distal end of the biopsy channel in order to exit from the biopsy channel and connect to the drying element. In certain embodiments, the drying device is removably attachable to a distal end portion of the advancing or navigation element. In other embodiments, the drying device is permanently attached to the navigation element. These embodiments allow the drying element to be translated from the distal end of the scope lumen to its proximal end or vice versa.

In certain embodiments, the drying device includes a programmable motor coupled to the elongate member and configured to translate the device through the lumen of the endoscopic instrument. The programmable motor is preferably configured to withdraw the elongate member through the lumen a specified distance for a specified duration of time.

In another aspect, a method for drying one or more lumens within an endoscopic instrument comprises providing a drying device comprising an elongate member and at least one drying member coupled to a portion of the elongate member and advancing at least one portion of the elongate member through the lumen within the endoscopic instrument such that the drying member substantially maintains contact with a wall of the lumen. In certain embodiments, the drying device is sterilized or disinfected so that the disinfected and/or sterilized device channel is not compromised by the drying process.

In embodiments, the method further comprises centering the drying member within the lumen as the drying member is advanced through the lumen. The centering element is preferably smaller than the diameter of the lumen through which the device is being advanced, but has a significant enough size to prevent misalignment and deflection of the navigation element to one side or another of the lumen as it navigates, including as the drying element is pulled or pushed around curves, corners and junctions of various lumens (including Y junctions).

The method may further comprise introducing a guidance element through a first opening in a lumen of an instrument and advancing an elongate drying device through a second opening in the lumen such that at least a portion of the elongate drying device engages the guidance element. The guidance element engages and couples with the drying device such that the drying device can be withdrawn towards the first opening of the lumen with the guidance element.

In certain embodiments, the endoscope lumen comprises first and second lumens coupled to each other at a junction, such as the Y junction between the biopsy and suction channels in an endoscope. In these embodiments, the method further comprises introducing the guidance element through the first lumen past the junction into the second lumen and advancing the elongate drying device through the second lumen such that the elongate drying device engages the guidance element. The guidance element may then be used to withdraw the drying device past the junction and through the first lumen. This ensures that the drying device dries the biopsy channel, rather than being deflected and continuing past the Y-junction proximally further into the suction channel.

In other embodiments, the endoscope lumen includes a turn or bend having a relatively small radius of curvature that would otherwise be difficult to advance the drying element therethrough. In these embodiments, the guidance element is introduced through a lumen on one side of the turn and advanced therethrough. The drying device is advanced or retracted through the lumen on the other side of the turn until it engages with the guidance element. The guidance element is then withdrawn to pull the drying device through the turn.

The guidance element may comprise a tubular sheath or similar device having a distal end portion configured for engaging an end portion of the cleaning element. In some embodiments, the tubular sheath is removably coupled to the cleaning element. In other embodiments, the tubular sheath may have an inner diameter larger than an outer diameter of the proximal end portion of the elongate drying element, and may be further configured to deflect or otherwise direct the drying element past a junction or other tortuous area in the lumen

In another aspect, a kit for cleaning and drying an endoscopic instrument comprises a drying device having an advancement element configured for advancement through a lumen within the endoscopic instrument and at least one drying member coupled to a portion of the advancement element. At least a portion of the drying member has an outer diameter equal to or greater than the outer diameter of the elongate member. The kit further comprises a cleaning device having a second elongate member configured for advancement through the lumen within the endoscopic instrument and at least one cleaning member coupled to a portion of the second elongate member. The cleaning member may be used to clean the lumen at an earlier stage of the process of cleaning and, in embodiments, disinfecting or sterilizing a device.

In one embodiment, the drying device is one of the embodiments described above and the cleaning member comprises distal and proximal end portions and a central portion between the distal and proximal end portions and wherein the central portion is shaped to create a pressure gradient along the central portion from the distal end portion to the proximal end portion. The variable pressure central portion of the cleaning device is designed to create variable pressure between the two circumferential elements and the wall of the channel being cleaned. Thus, as the cleaning member is advanced inside a channel and the scope and its channels are submerged in cleaning fluid (as required by scope manufacturers), the variable pressure design between the two circumferential elements creates a venturi effect between the cleaning element and the walls of the endoscope channel when the cleaning element is moved through the lumen. As a result, when the cleaning fluid flows across the variable pressure area, this impacts the fluid flows as it transfers from an area of high pressure across an area of low pressure and then back to another area of high pressure between the two cylindrical elements. This directs the cleaning fluid at the channel walls with an increased velocity and force, thereby removing more biomatter and other debris than conventional devices. This same pressure wave form effect may be created with the drying device where air is pressured and flows in a manner like the fluid flow of the liquid.

In another embodiment, the cleaning device may comprise any of the combinations of the elements described above with respect to the drying device.

In another aspect, a kit for cleaning and drying an endoscopic instrument comprises a drying/cleaning device having an elongate member configured for advancement through the lumen within the endoscopic instrument and at least one cleaning/drying member coupled to a portion of the second elongate member. In this aspect, the drying/cleaning device is configured to both clean and dry an internal lumen while being passed therethrough In embodiments, this version may be particularly useful to clean and dry after the use of liquid chemical sterilants and disinfectants, or one aspect of the kit may be used to clean and a separate aspect (preferably disinfected or sterile) may be used to dry.

In a preferred embodiment, the invention is configured for use with an endoscope. The advancing or navigation element is a polymer filament, wire or tube. The materials for the advancing element are any that are appropriate for the application, which, in embodiments, could be polypropylene, PET, nylon, PEEK, PTFE, ETFE, polyurethane, high or low density polyethylene, or other suitable polymer material. The advancing or navigation element could also be a metal wire (including stainless steel, nitinol or other material), or a combination of materials, including polymer reinforced by a wire, or a wire covered with a polymer or a combination thereof.

In one embodiment, the drying element is injection molded either over the advancing element or connected or glued to the advancing element. The injection molded drying element is any durometer that aids in drying the channel, though in a preferred embodiment, the durometer of the material is between 35 and 75 Shore A. In embodiments, the drying element may be made of any elastomer or any elastic material, including pebax, polyurethane, santoprene, silicone, high or low density polyethylene, EVA and other elastomeric materials. In embodiments, the drying element could be a combination of materials, including a combination of hard and soft materials with varying elasticity.

In embodiments, a version of the drying portion of the invention may include material that can absorb water, such as a sponge, an absorptive cloth or other material that can absorb and hold moisture and water. This material may be attached to the advancing element as a cylinder, as strips of material in singular or various lengths, as overlapping material, as a round ball, or as any other shape that aids in removable of water and moisture from a channel as the advancing element is passed through a channel. This absorptive material may be at the end or beginning of a drying element or interspersed between other drying elements, such as squeegees or fins.

The invention may include coatings that aid in attracting or repelling water and in navigation by changing levels of friction. The invention may have a lumen that allows air to be blown through the invention to delivery forced air deep inside a channel in close or direct proximity to a channel wall

In other embodiments, the cleaning/drying device may have one or more absorbent sponges placed in front of or at the end of the cleaning/drying device or in between one or more of the cylindrical elements to absorb biomatter and debris. The absorbent sponges may be of a single cell configuration or have multiple sponges with different cell configurations to provide scrubbing, absorption, lifting, diffusion of cleaning fluid, or a combination of these attributes. The absorbent sponges may comprise any material that absorbs biomatter, fluid or other debris, such as a polymer, foam, sponge, bamboo, hemp, microfibers, polyurethane, polyvinyl alcohol, or the like. In one embodiment, the cleaning member comprises a sponge-like material, such as cellulose, dry, natural and/or compressed cellulose. In an exemplary embodiment, the material comprises a mixture of cellulose and compressed cellulose that allows the sponge to expand when it is hydrated. Preferably, the material is selected such that the sponge has the ability to expand to at least the internal surface of the lumen, while, in a preferred embodiment, maintaining sufficient absorbability to absorb a volume of material or fluid at least equal to the volume of the segment of the lumen it occupies.

In embodiments, the sponges are soft and atraumatic when immersed in fluid, and expand to a size that is at least sufficient to remove debris from the channel being cleaned (and/or remove moisture and fluid from the channel), and in a preferred embodiment is larger than the channel being cleaned. The sponges may be any shape and size that conforms and aids in cleaning the scope's channel, including by way of example, not limitation, cylindrical in shape, spiral in shape, conical, triangular, square or any combination thereof, and can be disinfected or sterilized.

In other embodiments, the devices disclosed herein may be used to dry in-dwelling devices. In these embodiments, the devices may further include a sheath or similar structure to cover the drying elements and/or the elongate element while translating through the lumen of the instrument to avoid disturbing biomatter and any accumulated biofilm therein. The system may also include a measuring device, such as a marker on the elongate element or a separate element to confirm positioning of the device within the lumen of the catheter. The polymers that form the drying elements may expand once the sheath has been withdrawn. Alternatively, electrically responsive polymers may be used to change shape and enlarge with the application of energy, causing them to expand and contact the walls of the catheter, or at least in part.

The device may include a coating that is hydrophobic. In yet another embodiment, the device is superhydrophobic and/or oleophobic. In even still another embodiment, the device is anti-infective and hydrophobic. Further yet in another embodiment, the device is anti-infective and superhydrophobic. In further still another exemplary embodiment, anti-inflammatory coatings are incorporated into the device. In other embodiments, the anti-inflammatory coatings may comprise a hydrophilic material, or are hydrophilic.

It is to be understood that both the foregoing general description and the following detailed description are exemplary and explanatory only and are not restrictive of the description. Additional features of the description will be set forth in part in the description which follows or may be learned by practice of the description.

### BRIEF DESCRIPTION OF THE DRAWINGS

The accompanying drawings, which are incorporated in and constitute a part of this specification, illustrate several embodiments of the description and together with the description, serve to explain the principles of the description.

FIG. 1 illustrates a representative endoscope for use with the systems and methods disclosed herein;

FIG. 2 is a cross-sectional view of a representative endoscope, showing one or more lumens within the endoscope;

FIG. 3 is a side view of one end of a drying and cleaning device;

FIG. 4 is a side view of the other end of the drying and cleaning device of FIG. 3; and

FIG. 5 is a perspective view of another embodiment of a drying device;

FIG. 6 is a side view of an air drying device;

FIG. 7 is a side view of another embodiment of an air drying device;

FIG. 8 illustrates another embodiment of a cleaning and drying device; and

FIG. 9 illustrates a kit for cleaning and/or drying a beverage container.

### DESCRIPTION OF THE EMBODIMENTS

This description and the accompanying drawings illustrate exemplary embodiments and should not be taken as limiting, with the claims defining the scope of the present description, including equivalents. Various mechanical, compositional, structural, and operational changes may be made without departing from the scope of this description and the claims, including equivalents. In some instances, well-known structures and techniques have not been shown or described in detail so as not to obscure the description. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

While the following description is primarily directed to an endoscope and a device for drying the endoscope, it should be understood that the features of the presently described system may be readily adapted for use with a variety of reusable or disposable endoscopic instruments and devices that include internal lumens or other internal spaces, such as endoscopes, trocars, cannulas, dilatation devices, Foley catheters, guidewires, central venous catheters, bipolar or monopolar electrosurgical or ultrasonic devices, ventilation machines, respirators, CPAP and other positive airway machines, robotic and remote surgery equipment, arterial lines, drainage catheters, peripherally inserted central catheters, endotracheal tubes, feeding tubes, and other devices that in-dwell, penetrate and/or navigate in the body.

The term “endoscope” as used herein refers generally to any scope used on or in a medical application, which includes a body (human or otherwise) and includes, for example, a laparoscope, arthroscope, colonoscope, gastroscope, duodenoscope, endoscopic ultrasound scope, bronchoscopes, enteroscope, cystoscope, laparoscope, laryngoscope, sigmoidoscope, thoracoscope, cardioscope, and saphenous vein harvester with a scope, whether robotic or non-robotic, or in a non-medical application.

When engaged in remote visualization inside the patient's body, a variety of scopes are used. The scope used depends on the degree to which the physician needs to navigate into the body, the type of surgical instruments used in the procedure and the level of invasiveness that is appropriate for the type of procedure. For example, visualization inside the gastrointestinal tract may involve the use of endoscopy in the form of flexible gastroscopes and colonoscopes and specialty duodenum scopes with lengths that can run many feet and diameters that can exceed 1 centimeter. These scopes can be turned and articulated or steered by the physician as the scope is navigated through the patient. Many of these scopes include one or more working channels for passing and supporting instruments, fluid channels and washing channels for irrigating the tissue and washing the scope, insufflation channels for insufflating to improve navigation and visualization and one or more light guides for illuminating the field of view of the scope.

Smaller and less flexible or rigid scopes, or scopes with a combination of flexibility and rigidity, are also used in medical applications. For example, a smaller, narrower and much shorter scope is used when inspecting a joint and performing arthroscopic surgery, such as surgery on the shoulder or knee. When a surgeon is repairing a meniscal tear in the knee using arthroscopic surgery, a shorter, more rigid scope is usually inserted through a small incision on one side of the knee to visualize the injury, while instruments are passed through incisions on the opposite side of the knee. The instruments can irrigate the scope inside the knee to maintain visualization and to manipulate the tissue to complete the repair

Other scopes may be used for diagnosis and treatment using less invasive endoscopic procedures, including, by way of example, but not limitation, the use of scopes to inspect and treat conditions in the lung (bronchoscopes), mouth (enteroscope), urethra (cystoscope), abdomen and peritoneal cavity (laparoscope), nose and sinus (laryngoscope), anus (sigmoidoscope) and other aspects of the gastrointestinal tract (gastroscope, duodenoscope, colonoscope), chest and thoracic cavity (thoracoscope), and the heart (cardioscope). In addition, robotic medical devices rely on scopes for remote visualization of the areas the robotic device is assessing and treating.

These and other scopes may be inserted through natural orifices (such as the mouth, sinus, ear, urethra, anus and vagina) and through incisions and port-based openings in the patient's skin, cavity, skull, joint, or other medically indicated points of entry. Examples of the diagnostic use of endoscopy with visualization using these medical scopes includes investigating the symptoms of disease, such as maladies of the digestive system (for example, nausea, vomiting, abdominal pain, gastrointestinal bleeding), or confirming a diagnosis, (for example by performing a biopsy for anemia, bleeding, inflammation, and cancer) or surgical treatment of the disease (such as removal of a ruptured appendix or cautery of an endogastric bleed).

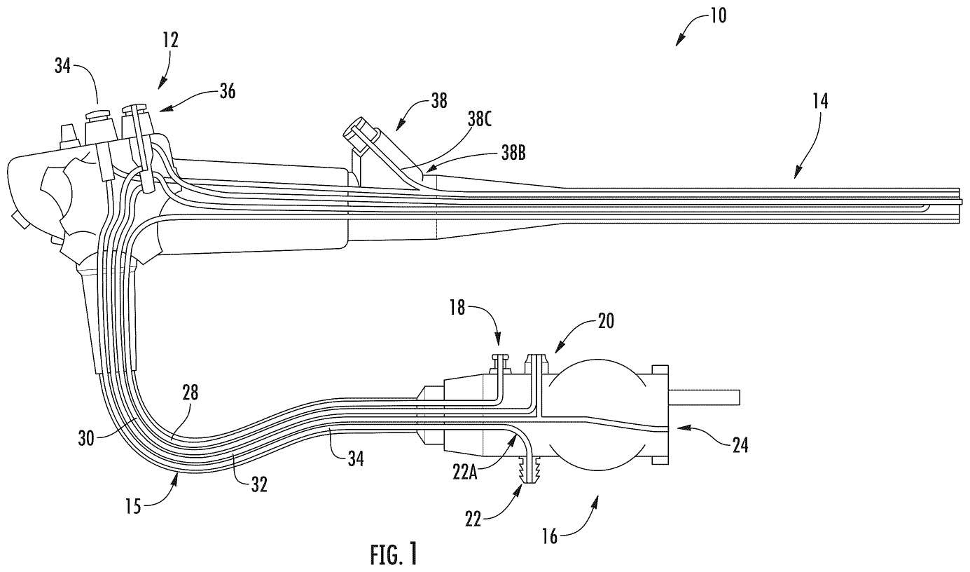

Referring now to FIG. 1, a representative endoscope 10 includes a proximal handle 12 adapted for manipulation by the surgeon or clinician coupled to an elongate shaft 14 adapted for insertion through an endoscopic or percutaneous penetration into a body cavity of a patient. Endoscope 10 further includes a fluid delivery system 16 coupled to handle 12 via a universal cord 15. Fluid delivery system 16 may include a number of different tubes coupled to internal lumens within shaft 14 for delivery of fluid(s), such as water and air, suction, and other features that may be desired by the clinician to displace fluid, blood, debris and particulate matter from the field of view. This provides a better view of the underlying tissue or matter for assessment and therapy. In the representative embodiment, fluid delivery system 16 includes a water-jet connector 18, water bottle connector 20, a suction connector 22 and an air pipe 24. Water-jet connector 18 is coupled to an internal water-jet lumen 28 that extends through handle 12 and elongate shaft 14 to the distal end of endoscope 10. Similarly, water bottle connector 20, suction connector and 22 air pipe 24 are each connected to internal lumens 30, 32, 34 respectively, that pass through shaft 14 to the distal end of endoscope 10.

Proximal handle 12 may include a variety of controls for the surgeon or clinician to operate fluid delivery system 16. In the representative embodiment, handle 12 include a suction valve 34, and air/water valve 36 and a biopsy valve 38 for extracting tissue samples from the patient. Suction channel 34 extends from suction connector 22, where it creates a relatively tight turn or bend 22A through universal cord 15 into handle 12. Suction channel 34 then extends through shaft 14 to the distal end of endoscope 10. As suction channel 34 passes biopsy valve 38, it creates an internal Y junction 38B with the channel 38C extending into biopsy valve 38. This Y junction 38b creates challenges for cleaning and/or drying suction channel 34 with conventional devices, as discussed in more detail below.

Handle 12 may in certain embodiments also include an eyepiece (not shown) coupled to an image capture device (not shown), such as a lens and light transmitting system. The term “image capture device” as used herein also need not refer to devices that only have lenses or other light directing structure. Instead, for example, the image capture device could be any device that can capture and relay an image, including (i) relay lenses between the objective lens at the distal end of the scope and an eyepiece, (ii) fiber optics, (iii) charge coupled devices (CCD), (iv) complementary metal oxide semiconductor (CMOS) sensors. An image capture device may also be merely a chip for sensing light and generating electrical signals for communication corresponding to the sensed light or other technology for transmitting an image. The image capture device may have a viewing end-where the light is captured. Generally, the image capture device can be any device that can view objects, capture images and/or capture video.

In some embodiments, endoscope 10 includes some form of positioning assembly (e.g., hand controls) attached to a proximal end of the shaft to allow the operator to steer the scope. In other embodiments, the scope is part of a robotic element that provides for steerability and positioning of the scope relative to the desired point to investigate and focus the scope.

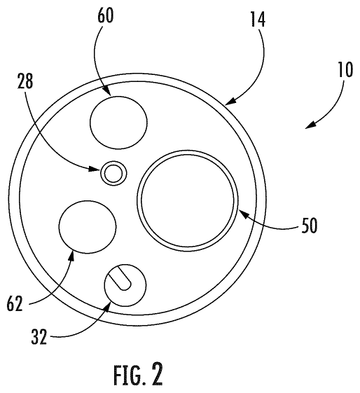

As shown in FIG. 2, endoscope 10 may further include a camera lens 62, a scope washer 32, and a light source 60 for providing a view of the surgical site in the patient, and a biopsy channel 50 for passing instruments therethrough. The biopsy channel 50 permits passage of instruments down the shaft 14 of endoscope 10 for removing tissue. Biopsy channel 50 may also function as a working channel for other instruments to pass through endoscope 10 for assessment and treatment of tissue and other matter. Such instruments may include cannulas, catheters, stents and stent delivery systems, papillotomes, wires, other imaging devices including mini-scopes, baskets, snares and other devices for use with a scope in a lumen. Alternatively, endoscope 10 may include a separate working channel for these instruments.

An exemplary drying device will now be described. The cleaning drying device comprises an elongate shaft and a drying member disposed on one portion of shaft. The drying member may be removably attached to, or permanently affixed to, the shaft. The shaft may comprise any suitable material that provides sufficient rigidity for the shaft to be advanced through a lumen of an endoscope. The elongate shaft has an outer diameter sized to fit within, and translate through, the internal lumens in endoscope 10. In the exemplary embodiment, the shaft will have an outer diameter in the range of about 0.5 to about 5 mm, preferably about 1 to 4 mm.

The drying members may comprise one or more substantially cylindrical members spaced from each other along the advancement member. The cylindrical members are configured to maintain contact with the channel wall to displace and remove any moisture as the advancement element travels through the channel. The advancement element may be advanced through being pushed, pulled or by other means, including back and forth agitation, advancement and withdrawal based on a pattern. The advancement element may travel through the scope by connection to an automated or mechanized approach or any other means of moving the channel drying element through the channel to displace, absorb and or remove water at least in part through direct contact with the surface area to be dried.

In certain embodiments, the drying devices described herein are sterilized or disinfected so that the disinfected and/or sterilized device channel is not compromised by the drying process. Instead, when the drying device is passed through the already disinfected or sterilized device channel, water and moisture is removed without altering or contaminating the disinfected or sterilized state of the device with the channel.

In certain embodiments, the device includes a pull cable configured to withdraw or advance elongate the shaft within an internal lumen in endoscope 10. Device may also include an energy source and a motor for advancing and/or withdrawing the elongate shaft. Of course, it will be recognized that the elongate shaft may be manually translated through internal lumen via a proximal handle or suitable actuator (i.e., no motor).

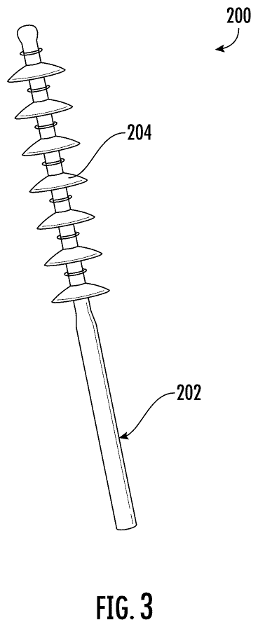

Referring now to FIG. 3, a drying device 200 according to certain embodiments will now be described. Drying device 200 include an advancement element 202 and one or more drying element(s) 204, which are attached to advancement element 202. Advancement element 202 can be advanced from the proximal end to the distal end (or vice versa) of any internal lumen with the endoscope. For example, in one embodiment, advancement element 202 is advanced from the scope's biopsy channel to the distal end of the biopsy channel in order to exit from the biopsy channel and connect to a cleaning and/or drying element. Advancement element 202 may also be advanced from the proximal end of the scope's biopsy channel to the distal end of the biopsy channel (or suction channel, as applicable) in order to exit from the biopsy channel and connect to a drying element, or alternatively, advanced and pulled through the channel from the proximal end of the biopsy channel (or suction channel) to the distal end. This pushing and/or pulling advancement element is attachable and in embodiments also detachable, and in other embodiments may be permanently attached to the drying element.

In other embodiments, advancement element 202 and drying elements 204 are adhered to each other and advanced or retracted through one or more lumens together. Advancement element 202 and drying elements 204 may be manufactured as one integral device, or they may be manufactured separately and attached to each other prior to use.

Drying elements 204 may comprise any suitable shape that substantially conforms to the walls of the internal lumen, thereby creating a consistent circumferential contact with the interior wall of an endoscope channel, such as a biopsy or suction channel, to remove fluid and moisture from the channel. In certain embodiments, drying elements 204 preferably have a substantially annular, circumferential, cylindrical or conical shape and having a diameter approximately equal to or slightly larger than the diameter of the internal lumen. In an exemplary embodiment, the largest diameter of drying elements 204 is about 1 to about 1.5 times the diameter of the internal lumen, preferably about 1 to about 1.23 times this diameter. For example, if the diameter of the internal lumen is about 5 mm, the largest diameter portion of elements 204 may be about 4.2 to 5.5 mm, preferably about 5 mm. This additional size allows elements 204 to deform slightly as they pass through the lumen, ensuring that they will remain in contact with the lumen.

Drying elements 204 may be arranged in any manner that is effective at removing fluid and moisture from the channel. This includes in groups of two and then three squeegees, or vice versa, or in other configurations of squeegees, such as groups of one and then two squeegees, two and then four, three and three, four in a row, five in a row or any other number and configuration of squeegees that is effective for the removal of water and moisture, depending on the channel diameter, length, curves, junctures with other channels and other factors.

In embodiments, certain of the drying elements 204 may be of different heights or outer dimensions in order to make the device 200 more adaptable to use in channels of different sizes. For example, the device may have a drying element 204 that is 5 to 5.2 mm in diameter followed by a smaller drying element 204 that is between 2 to 2.2 mm in diameter, followed by another drying element 204 that is 3.8 to 4 mm in diameter to create variable wall contact across a variety of channel sizes. In embodiments, the height and arrangement of drying elements 204 is not limited to this description and these diameters, placements, drying elements 204 thickness and other shall vary based on what is most effective for the drying for a given channel drying application.

The spacing between drying elements 204 may be any spacing that supports rapid and predictable channel drying, including a spacing of 1 mm, 1.25 mm, 1.5 mm, 2 mm, close spacing of 1.25 mm and then a 4 mm gap and then spacing of 1.25 to 1.5 mm or other spacing closer or farther or more variable that optimizes and supports rapid and predictable channel drying. Spacing, diameters and alignment and grouping of drying elements may vary based on channel size, channel material, the number of curves to navigate around, the number of junctions with other channels, if any, the channel dryer material, and other factors specific to the given channel drying application, in embodiments.

The thickness of drying elements 204 are any thickness that aids in channel drying and the positioning of the elements 204 to dry. In embodiments, this includes drying element thickness of between 0.2 and 0.5 mm, other variants with a thickness of 0.5 to 1.0 mm; other variances with tapered thickness with the thickness greater at the base of the drying element and tapering closer to the channel wall to enhance deflection and conformance of the element to the channel wall, while providing support and a certain rigidity and flexibility around the advancing element.

In embodiments, device 200 includes a centering element (not shown), so that the drying elements 204 remains centered as they are advanced through the channel to dry. This centering element prevents a drying element from losing contact with the wall of the channel being cleaned, enhancing drying action. The centering element may comprise any shape that keeps the drying elements in the relative center of the channel, so that any lateral movement needed to navigate through the channel, especially around curves, does not cause the drying element to flatten against a wall and thereby lose contact with the channel wall it is drying as it is advanced. In an exemplary embodiment, the centering element is between 50% and 95% of the diameter of the channel being dried or cleaned. The centering element may be placed anywhere on the advancing element or the drying elements, including multiple centering elements to enhance and preserve drying and cleaning performance.

In some embodiments, the centering element may comprise a series of shaped elements projecting from the elongate member or the navigation member. This series of shaped elements may, for example, comprise struts, spikes, or other projections extending radially outward from the elongate member and sized to substantially center the elongate member and/or the drying/cleaning elements within the internal channel of the endoscopic device.

The centering element(s) serve to center advancement element 202 and the drying device as the device is pulled or pushed through lumens around turns and navigates through corners and other complex areas, including junctions of multiple lumens and internal channels in the scope or other instrument being cleaned. The centering element(s), in embodiments, may be smaller than the diameter of the lumen through which the device is being advanced, but have a significant enough size to prevent misalignment and deflection of advancement element 202 to one side or another of the lumen as it navigates, including as the cleaning and/or drying device is pulled or pushed around curves, corners and junctions of various lumens (including Y junctions).

The centering element(s) may be any shape that keeps the device generally centered and prevents this deflection, with a preferred embodiment being a cylindrical shape with a tapered distal end. When this sort of misalignment occurs, which is an issue with existing brushes and pull thru cleaners, the brushes and other elements are pulled to one side of the lumen as the cleaners are pulled around curves, corners and junctions of lumens, with the result being contact with the lumen wall and the cleaning and/or drying element (whether a brush, pull thru or other cleaner) is minimized, adversely changed, or lost, resulting in an adverse impact on the effectiveness of the cleaning and/or drying approach. By placing a centering element at the front or back, or both of the device, this issue is corrected, resulting in more consistent, effective cleaning and/or drying, especially around curves, corners, channel junctions and other complex areas inside an endoscope or other endoscopic instrument or device.

In a preferred embodiment, the centering element(s) are between 50 percent and 90 percent of the diameter of the lumen being cleaned, with a further preferred embodiment having a diameter or height between 70 percent and 85 percent of the diameter of the lumen being cleaned. The entering element(s) can be any shape that preserves the centering of the cleaning and/or drying element as it is navigated through a channel. In embodiments, this includes cylindrical, conical, spherical and a centering element may be placed at the distal area of the device, at the distal and proximal end, between cleaning and/or drying members, or the proximal end, as appropriate to aid in centering the cleaning and/or drying element, especially as it navigates around curves, across Y-junctions and other aspects of a lumen.

The materials for advancement element 202 can be any material sufficient to navigate through the channel being cleaned and able to manage the pull force associated with advancing cleaning and/or drying element 202 through the channel being cleaned and dried. This includes all metal and polymer based materials, including stainless steel wires, nitinol, and other metals. It also includes all polymer based materials, whether in a monofilament form, extruded tube, braided or any other form sufficient to facilitate advancing the cleaning and/or drying element 204 though the channel being cleaned and dried. In a preferred embodiment, advancement element is a monofilament composed of nylon, polyamide, polyurethane or other polymeric material, with a diameter of at least 1 mm. Advancement element 202 may include a grip element (not shown) at one end that facilitates holding and passing advancement element 202. In embodiments, this grip element is larger than the entry point to the biopsy channel to protect against over advancing advancement element 202 into the biopsy channel and losing one's grip on advancement element 202.

In certain embodiments, advancement element 202 is attachable to drying elements 204 through permanent attachment, which can be through molding, overmolding, two shot molding, glue or other means to create an attachment between the advancement and drying elements where the two element are fixed or affixed for use. Alternatively, advancement element 202 may be separately attachable and in certain embodiments attachable and detachable, so that advancement element 202 may be attached from one end of a channel the other end, exit the channel and then be attached to drying elements 204. The means of attachment is any way suitable for the intended use, which by way of example may include interlocking elements, compression fitting, a slide and locking mechanism, a loop and a hook mechanism, an insert and twist mechanism or variations and alternative combinations suitable for the diameter and shape of the navigation and cleaning and/or drying devices.

In embodiments, drying device 200 may comprise one or more absorbent sponges placed in front of or at the end of drying elements 204 or in between one or more of the drying elements. The absorbent sponges may be of a single cell configuration or have multiple sponges with different cell configurations to provide scrubbing, absorption, lifting, diffusion of cleaning fluid, or a combination of these attributes. The absorbent sponges may be of any material, including polyurethane, polyvinyl alcohol, or other absorbent material. In embodiments, the sponges are soft and atraumatic when immersed in fluid, and expand to a size that is at least the size of the channel being dried, and in a preferred embodiment is larger than the channel being dried. The sponges may be any shape that conforms and aids in drying the scope's channel, including by way of example, not limitation, cylindrical in shape, spiral in shape, conical, triangular, square or any combination thereof. In an exemplary embodiment, the sponge(s) will have a pore size of between about 200 to 1500 PPC, preferably between about 200 PPC and about 600 PPC.

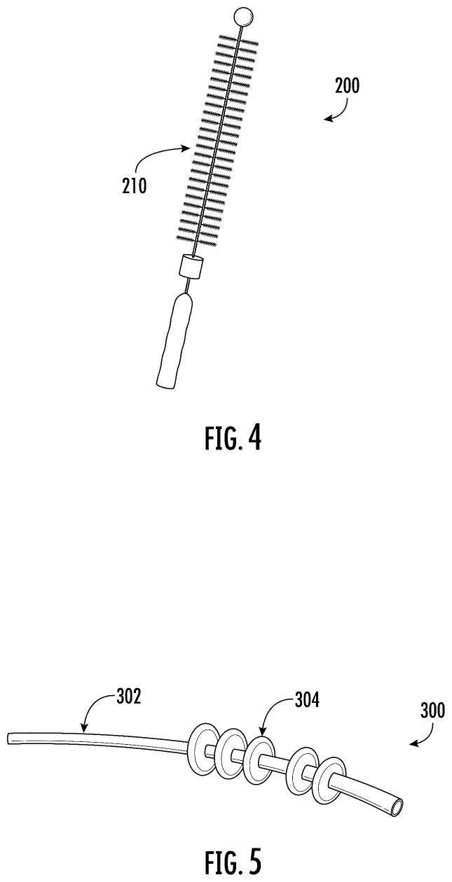

Referring now to FIG. 4, cleaning device 200 may include a brush 210 of various designs, such as a tapered nylon brush, which contacts a portion of the channel wall in addition to the other aspects of drying element 200. In one embodiments, brush 210 is located on an opposite end of advancement element 202 from drying elements 204. This allows the user to, for example, clean an endoscopic lumen or channel with brush 210 and then dry the channel with drying elements 204. The brush may be of a length, for example, in the ratio of 1.0 to 1.4 times the diameter of the channel to be cleaned. In embodiments, the brush is preferably made of an atraumatic polymer, such as polyurethane, with a thickness and durometer designed to limit trauma and injury to the channel wall, while maintaining sufficient rigidity to remove contamination from the walls of the channel. The diameter of the brush elements contacting the channel wall may be any diameter, but in embodiments may be between 0.5 and 2 mm. The brush elements may be perpendicular to the advancement element and in embodiments, may be part of a separate, shorter navigation element designed to reach only a few a limited distance into the biopsy channel. This shorter version may be any length appropriate for cleaning the initial entry points into the biopsy channel, but in a preferred embodiment is between 4.5 and 15 cm long. This brushing element, whether part of the cleaning/drying device or in a separate shorter version, may also utilize nylon wire bristles or other bristles if arranged in a pattern that is effective in cleaning and minimizes trauma to the scope channel. A grip element of the brush may have a shape at one end or in the center of the element that is larger to facilitate introduction into the biopsy channel.

Referring now to FIG. 5, another embodiment of a drying device 300 comprises an advancement element 302 and one or more drying elements 304. Advancement element 302 can be advanced from the proximal end to the distal end (or vice versa) of any internal lumen with the endoscope. In some embodiments, advancement element 302 and drying elements 304 are adhered to each other and advanced or retracted through one or more lumens together. Advancement element 302 and drying elements 304 may be manufactured as one integral device, or they may be manufactured separately and attached to each other prior to use.

Drying elements 304 may comprise any suitable shape that substantially conforms to the walls of the internal lumen, thereby creating a consistent circumferential contact with the interior wall of an endoscope channel, such as a biopsy or suction channel, to remove fluid and moisture from the channel. In certain embodiments, drying elements 304 preferably have a substantially annular, shape and have a diameter approximately equal to or slightly larger than the diameter of the internal lumen.

The spacing between drying elements 304 may be any spacing that supports rapid and predictable channel drying, including a spacing of 1 mm, 1.25 mm, 1.5 mm, 2 mm, close spacing of 1.25 mm and then a 4 mm gap and then spacing of 1.25 to 1.5 mm or other spacing closer or farther or more variable that optimizes and supports rapid and predictable channel drying. In one embodiment, drying device 300 comprises a first group of three drying elements 204 spaced from each other by a substantially equal distance and a second group of two drying elements 204 also spaced from each other by a substantially equal distance. The first and second groups of drying elements 204 may be spaced from each other by the same distance as the individual elements, or by a larger distance (as shown in FIG. 5).

Drying element 300 may be effectively used to clean lumen channels having an internal diameter from about 2.8 mm to about 5.0 mm, or from about 1.4 mm to about 2.6 mm, or from about 1.0 mm to about 1.2 mm.

In another embodiment, a drying device may include a rod or filament like member having at least one end that terminates in a loop and a swab member attached to the loop. The swab member may have first and second ends where the first end is attached to the loop and the second end comprises a tail such as the tail of a kite where the tail can be of any length. The swab member may comprise one or more materials that can be braided or twisted with the tail end of the swab member comprising the unbraided or untwisted ends of the material or materials that comprise the swab member. In addition, the swab member may comprise a foam material and/or a microfiber material.

The rod member of the cleaning device may comprise a polymer having first and second ends where the first end is a straight end and the second end is a looped end. In addition, the rod member may comprise one continuous polymer rod with the looped end of the rod member comprising one thickness or diameter and the straight end of the rod member comprising two thicknesses or two diameters of the looped end of the rod. The two thicknesses or diameters of the polymer rod that form the straight end of the rod member may be fused or bonded together, laminated together, or co-extruded. A more complete description of a suitable kite-tail drying member can be found in U.S. Patent Publication No. 2014/0250614, the complete disclosure of which is incorporated herein by reference for all purposes.

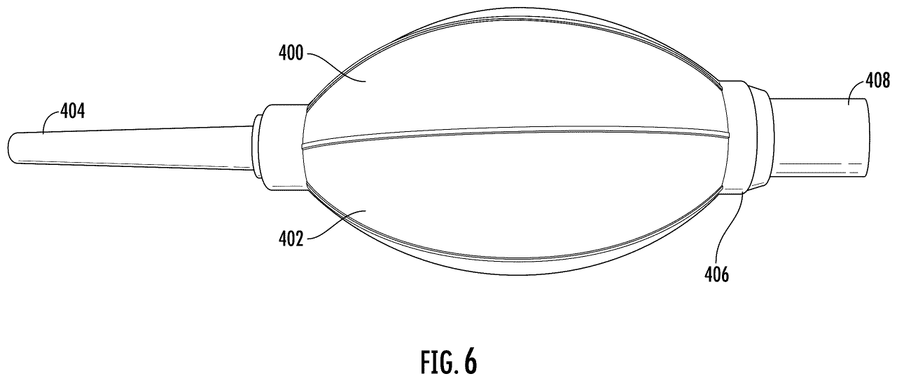

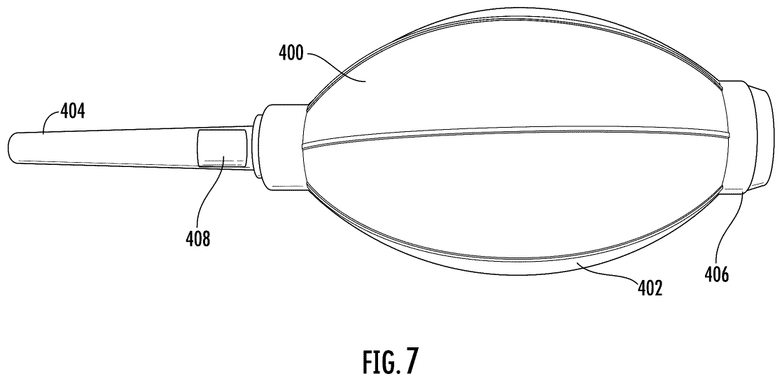

Referring now to FIGS. 6 and 7, another embodiment of a drying device 400 comprising a hand pump 402 coupled to an air nozzle 404 for delivering air to the endoscope channel and an air induction valve 406 for receiving air into the hand pump 402. Air nozzle 404 is preferably sized to fit within one of the valves of the endoscopic device, such as an air/washer valve. In embodiments, device 400 may include one or more detachable fittings or guidance elements to be inserted in the end of a channel for drying to create a seal to aid in forcing the air into the channel. Device 400 allows an operator to rapidly flush endoscopic channels with air without the need to use a motorized fan or central air line to force and delivery air into a channel.

Device 400 may further include a filter 408, such as a Hepa filter or the like, to filter the air that enters pump 402. In one embodiment, filter 408 is located adjacent air induction valve 406 (FIG. 6). In another embodiment, filter 408 is located within air nozzle 404 (FIG. 7).

Drying device 400 allows rapid and low cost delivery of air to flush a channel, which is beneficial for smaller channels and for accelerating the drying process further in connection with the drying devices described above. Device 400 may be sterilized or disinfected, or may include a sterile sheath or cover to hold pump 402. Pump 402 may be reusable, or single use, and may be sterilized, including with gamma, ETO, ebeam, steam and other forms of sterilization.

The materials for the hand air pump and related components may include one or more elastic and elastomeric materials. As an alternative embodiment, the rapid air pump may use a syringe or hand pump to push air into the channel to be dried, or may have a connector to access air from another source, such as a built-in source in the surgical unit, to direct this air through the heap filter and then the nozzle and into the scope channel. In embodiments, this connector may have a single air source one end a bifurcation that splits the air between multiple channels to dry. In embodiments, this may also include a valve to prevent backflow and to prevent air intended for one channel from crossing over to an adjacent channel in instances when the resistance to air flow varies between two adjacent channels and a common forced and/or filtered air source.

As an alternative to sterilizing the hand air pump, the innovation may include a sterile sheath or cover to insure the hand air pump is insight a sterile cover to deliver the forced air to the channel.

The devices describe above may be kitted with one or more related elements, including for example, but not limitation, a separate channel cleaner, a channel cleaning confirmation test (such as an ATP test), a sponge for cleaning, a scope carrier, enzymatic cleaning, detergent, a drying sponge or other hand drying element, a channel dry confirmation test, and other related products. A kit may also include a Tyvek or other bag or container that can be sealed to keep a dry medical device dry and preserve disinfection or sterilization. This bag or container may include material attached inside the bag or in a small pouch or other means that absorbs atmospheric humidity inside the bag or container. This bag or container and other elements of this kit may be sterile, disinfected or non-sterile.

A kit is also provided for use in cleaning and/or drying an endoscopic instrument. The kit includes any of the cleaning and/or drying devices described above and may include an endoscope instrument or an endoscope, such as any of the endoscopes described above in reference to FIG. 1, or others known by those skilled in the art. In addition, or alternatively, the kit may include a variety of other devices used for cleaning and/or drying procedures in any combination, such as cleaning brushes, swabs and/or sponges, enzymatic cleaners, disinfectants, and other devices and agents for sterilizing and/or disinfecting medical devices, scope drying agents, test strips or other sensors for determining the effectiveness of such cleaning and/or drying devices (i.e., detecting the presence of proteins, biomatter, bacteria, fungi, viruses, protein, ATP or bacteria markers, or other pathogens), personal protective equipment (PPE), scope housings for transporting scopes to and from, for example a reprocessing location, contamination bags and the like.

Another kit provided herein includes a cleaning device and a drying device. In certain embodiments, both the cleaning and drying devices may comprise one or more of the embodiments described above. The kit may include a non-sterilized or cleaning device and a sterilized drying device. Alternatively, the kit may include one device that functions to both clean and dry an internal lumen of an endoscopic device. In embodiments, a cleaning device may be one color and a drying device may be a different color in order for users to easily determine the purpose of each device. This aids in identifying the use of each device and it aids in making clear which device is sterilized after it is opened. The color difference may be the navigation element, the cleaning/drying element, a pad print line, or other marker to make clear the different uses of each device.

In certain embodiments, the kit includes a cleaning device such as one the embodiments described above and a drying device that comprises an advancement element that may be a filament, wire, tube or other element capable of advancement into an internal channel, including a long, curved, or other channel that is difficult to access because it is recessed or internal. In embodiments, this element can be grasped at an end once it is pulled through the internal channel and then pulled through the channel as part of the drying process. It also can be advanced from the other end through pushing the advancement element. This element may include a version that is able to advance air through the device and into the channel, including to a removed location well inside the channel and beyond the typical effectiveness of forced air drying in a channel.

In certain embodiments, the kit may include a drying device such as those described above, and a cleaning device that includes a variable pressure region. The variable pressure central portion is designed to create variable pressure between the two circumferential elements and the wall of the channel being cleaned. Thus, as the cleaning member is advanced inside a channel and the scope and its channels are submerged in cleaning fluid (as required by scope manufacturers), the variable pressure design between the two circumferential elements creates a venturi effect between the cleaning element and the walls of the endoscope channel when the cleaning element is moved through the lumen. As a result, when the cleaning fluid flows across the variable pressure area, this impacts the fluid flows as it transfers from an area of high pressure across an area of low pressure and then back to another area of high pressure between the two cylindrical elements, or in embodiments, from low pressure to high pressure and back to low pressure. This directs the cleaning fluid at the channel walls with an increased velocity and force, thereby removing more biomatter and other debris than conventional devices. A more complete description of such a cleaning device can be found in commonly assigned U.S. patent application Ser. No. 17/509,304, filed Oct. 245, 2021, the complete disclosure of which is incorporated herein by reference in its entirety for all purposes.

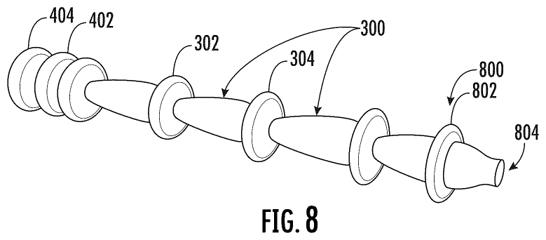

FIG. 8 illustrates another embodiment another embodiment of a cleaning and/or drying device 800. As shown, device 800 comprises multiple cleaning and/or drying elements 300 with each cleaning and/or drying element 300 including proximal and distal end portions 302, 304 and a variable pressure region 306 therebetween, as described above. Proximal and distal end portions 302, 304 are preferably cylindrical elements having an outer diameter substantially the same as the inner diameter of the lumen to be cleaned (as discussed above). In this embodiment, the cleaning and/or drying elements are coupled to each other at the proximal and distal end portions.

Variable pressure region 306 comprises a contraction section coupled to the proximal contact element 302, a diffusion section coupled to the distal contact element 304 and a throat section coupling the diffusion and contraction sections. The throat section has a diameter less than the diameter of the contact elements 302, 204 and greater than a diameter of the diffusion and contraction sections. This design enhances the performance of the cleaning fluid (or air) by turning the fluid (or air, as applicable) from a static point of interaction with the walls of a scope channel, to a dynamic point of interaction where the lifting action of the cleaning fluid's chemistry (or air in the case of drying) is enhanced through cleaning and/or drying member's direction of the fluid or air at the walls of the scope channel with pressure.

Variable pressure region 306 may include an inverted, partial venturi shape, a parabolic shape, a variable slope shape or such other shape that creates variable pressure between the two cylinders and the wall of the channel being cleaned, thereby increasing the force by which the cleaning fluid or air is projected at the channel wall when the cleaning and/or drying member is advanced.

In an exemplary embodiment, the throat section is substantially cylindrical. The contraction section preferably increases in diameter from the contact section 302 to the throat section and the diffusion section preferably decreases in diameter from the throat section to contact section 304, thereby creating a venturi effect between the distal and proximal end portions 302, 304 of the cleaning and/or drying element 310.

In a preferred embodiment, variable pressure region 306 has an inverted, partial venturi shape with three distinct areas of various spacing from the wall of the scope channel, which creates accelerated hydrodynamic action or similar action with air to project the cleaning fluid (or air) at the channel wall to clean more effectively. These areas include a contraction section, which is the start of the area where cleaning fluid (or air) is present on the other side of the first cylindrical element. The contraction section is the start of the area in which fluids accumulate and are subject to changing pressure as the space available for the fluid varies and becomes smaller as cleaning and/or drying element 300 is advanced and the fluids (or air) are directed into the throat section that further alters the pressure between the cleaning and/or drying element and the channel wall. The throat section, wherein the shape available for the fluid is reduced further in a manner that changes the pressure on the fluid (or air) compared to the pressure on the fluid (or air) in the contraction section, creates an acceleration of the fluid as cleaning and/or air as the drying element 300 is advanced; followed by a diffusion section which supports the diffusion of the cleaning fluid (or air) at an accelerated speed as it exits the throat section. Collectively, these sections between the cylindrical elements create a hydrodynamic force for cleaning fluids sufficient to remove bacteria, biomatter and debris from the walls of the channels of the endoscope or a similar force with air to displace water and moisture to dry the channel of the endoscope. This hydrodynamic force applied to air is also sufficient to remove substantially all moisture and fluid from the lumens during a drying process.

The angle of the slope of the contraction section (defined as the angle made between the vertical section of conical section 302 and the sloped portion of contraction section) may vary depending on the diameter of the channel being cleaned, the viscosity of the fluid (or thickness of air) and other factors and should be sufficient to support a variable pressure flow of cleaning fluid or air between the cylinders when the cleaning and/or drying element is advanced. In certain embodiments, the contraction section defines an angle with the proximal end portion (i.e., contact section 302) that is about 4 degrees to about 85 degrees, preferably between about 15 degrees to about 30 degrees. Similarly, the diffusion section defines an angle with the distal end portion (i.e., contact section 304) that is about 4 degrees to about 85 degrees, preferably about 15 degrees to about 30 degrees. Of course it will be recognized that various pressure regions 306 may have more than one slope, a curved shape, a variable shape or such other shape which assists in varying the pressure between the two cylindrical elements 302, 304.

Likewise, the angle between contraction and diffusion sections and throat section may vary depending on the diameter of the channel being cleaned, the viscosity of the fluid or air and other factors, and should be sufficient to support a variable pressure flow of cleaning fluid or air between the cylinders when the cleaning and/or drying element is advanced. In certain embodiments, this angle is about 10 degrees to about 50 degrees, preferably about 15 degrees to about 30 degrees and more preferably about 20 degrees to about 25 degrees.

The length and diameter of each section of variable pressure region 306 are preferably selected to optimize the venturi effect and will vary based on the diameter of the internal lumen, the viscosity of the fluid or air and other factors. For example, in a lumen having a diameter of about 4.2 mm, the length of throat section 310 may be about 2 mm to 10 mm, preferably about 3 mm to 5 mm, and more preferably about 4 mm. The outer diameter of throat section will also depend on the diameter of the inner lumen as well as the diameter of contraction and diffusion sections. In certain embodiments, throat section is less than the diameter of the internal lumen, but greater than 50% of the diameter of the lumen, preferably greater than about 60% of the diameter of the lumen, and more preferably equal to or greater than about 70% of the diameter of the lumen (e.g., about 3 mm in a lumen having an inner diameter of about 4.2 mm).

The venturi effect created by variable pressure region 306 impacts the fluid flows (and air flows, as applicable) as it transfers from an area of high pressure across an area of low pressure between the two cylindrical elements, and then back to another area of high pressure, such that the cleaning fluid or air is directed at the channel walls with an increased force, similar to the venturi effect created when putting one's thumb partially over the end of a garden hose to increase the force of the water emitting from the hose. This variable pressure design means that when cleaning and/or drying element 300 is attached and withdrawn or pulled with the navigation element through a scope channel, the cleaning fluid or air in the channel and between the cylindrical elements and the wall of the endoscope channel is moved across the variable pressure area between the two cylindrical spheres, creating a jetting of the cleaning fluid to pressure wash the channel walls of the endoscope channel with the cleaning fluid, or pressure of air to dry the channel walls.

This unique capability has the powerful effect of enhancing the performance of the cleaning fluid by turning the fluid from a static point of interaction with the walls of a scope channel, to a dynamic point of interaction where the lifting action of the cleaning fluid's chemistry is enhanced through the cleaning and/or drying element's direction of the fluid (or air) at the walls of the scope channel with pressure. Computational modeling using fluid and pressure dynamics shows that, in embodiments, the application of inverted venturi principles to create variable pressure between two cylindrical elements directs the cleaning fluid (or air) at all of the channel wall with hydrodynamic (or air) pressures of variable and increasing force to create a new, highly effective cleaning and/or drying capability that can remove debris, biomatter and bacteria from the channel, or dry, including addressing changes in the surface topography of the channel due to the ability to direct the cleaning fluid with hydrodynamic force (or air) into any scratches and crevasses in the scope channel.

In embodiments, this variable pressure region 306 causes the cleaning fluid or air to be projected at the channel wall with a pressure that exceeds the adhesion force of bacteria or moisture that may attach to the wall, creating a powerful benefit that is not present with existing brushing technologies. This capability enhances cleaning or drying, just as using a detergent with a pressure washer enhances the cleaning of an external surface, such as using a pressure washer with detergent to remove contaminants from the side of a building or a walkway, or an air dryer. This innovation enhances the cleaning fluid or drying in a new and powerful way, plus adds other capabilities in its design, changing channel cleaning performance so that the successful cleaning of a scope channel is not dependent on the performance of a single element, such as the unpredictable wall contact force of a bristle brush or a pull thru cleaner, or the static performance of a cleaning detergent, or drying based on evaporation over time. The variable pressure region 306 of cleaning/drying element 300 creates hydrodynamic/air pressure that directs cleaning detergent/air at the wall of the scope's channels. Cleaning or drying action using the hydrodynamic pressure force to enhance cleaning fluid performance or air drying and doing this in combination with mechanical pressure force is the best way to achieve consistent, predictable and repeatable success with removing biomatter and debris or moisture through drying from the channels of endoscopes, or other endoscopic instruments, without injury to these important channels.

The combination of cylindrical elements and a variable pressure element is important for creating the hydrodynamic or air force and it adds additional cleaning and/or drying force, by making atraumatic contact with the walls of the scope channel. These cylindrical elements add a channel wall contact pressure force as an additional cleaning or drying modality to remove debris and biomatter from the channel wall or to dry as an additional, complementary cleaning or drying capability that works in concert with the variable pressure element between the cylinders.

One embodiment of a cleaning and/or drying device 800 with multiple cleaning and/or drying elements 300 will now be described. As shown, each cleaning and/or drying element 300 includes proximal and distal end portions 302, 304 and a variable pressure region 306 therebetween, as described above. Proximal and distal end portions 302, 304 are preferably cylindrical elements having an outer diameter substantially the same as the inner diameter of the lumen to be cleaned (as discussed in detail below). In this embodiment, the cleaning and/or drying elements are coupled to each other at the proximal and distal end portions. In an exemplary embodiment, the proximal end portion of one cleaning and/or drying element is integral with the distal end portion of the next cleaning and/or drying element, although it will be recognized that other configurations are possible. For example, cleaning and/or drying device 400 may have more than one cylindrical element placed in close proximity to another cylindrical element with a spacing that does not create variable pressure, followed by or, alternatively, before, a cylindrical element with a spacing between the next cylindrical element that creates variable pressure between cleaning and/or drying element 300 and the wall of the channel being cleaned. In embodiments, a series of cylindrical elements may be organized in various spacing to create variable pressure between the cylindrical elements and certain spacing to create constant pressure between the cylindrical elements.

Cylindrical elements 302, 304 may be made of any shape and size that makes contact and conforms at least in part to the walls of the channel being cleaned, including in embodiments, cylindrical elements with a taper, a reverse taper, cylindrical elements that deflect and contact each other or which deflect and do not contact another cylindrical element, or which contact or do not contact a variable pressure shape between the cylindrical elements. The cylindrical elements do not have to be cylindrical, but need to be able to assist with creating a variable pressure result with the rest of the elements of cleaning and/or drying device 400, which means they must have wall contact that is meaningful enough to support creating a variable pressure area to accelerate fluid flow or air flow and thereby direct the cleaning fluid or air at the channel wall with hydrodynamic or air pressure.

Cleaning and/or drying device 800 includes two additional cylindrical elements 402, 404 at a distal end of the device 800. In this embodiment, device 800 further includes a proximal tip element 802 and a proximal cylindrical centering element 804 disposed around variable pressure region 306. Element 804 is preferably disposed around the contraction section 308 of pressure region 306 between the diffusion section 312 and the throat section 310 (see FIG. 4 and the above description of these elements).

Centering element 804 is positioned around pressure region 306, rather than at the proximal tip of the device (as in the FIG. 7B embodiment). Centering element 804 is sized such that its outer surface contacts the internal surface of a lumen of an endoscopic device. As device 800 is pulled or pushed through the lumen, centering element 804 engages the internal surface of the lumen and displaces any biomatter (cleaning) and/or moisture (drying) on this surface and moves that material forward along with device 800. In the event that the d FIG. 27 illustrates a kit 900 that includes a water bottle 902 having a straw 904 and a cleaning and/or drying device 910 for the straw 904. In this embodiment, device 910 may be used to clean and/or dry the internal surfaces of drinking containers and components of drinking containers, such as drinking straws, beverage containers, water containers, bottles, thermoses, canteens, jugs, carafes, baby bottles, water bottles and insulated beverage containers and drinking straws for any of these containers.

As shown, device 910 comprises multiple cleaning and/or drying elements 300 with each cleaning and/or drying element 300 including proximal and distal end portions 302, 304 and a variable pressure region 306 therebetween, as described above. Proximal and distal end portions 302, 304 are preferably cylindrical elements having an outer diameter substantially the same as the inner diameter of the lumen to be cleaned (as discussed above). In this embodiment, the cleaning and/or drying elements are coupled to each other at the proximal and distal end portions. Cleaning and/or drying device 910 includes two additional cylindrical elements 402, 404 at a distal end of the device 910.

In this embodiment, device 910 further includes a proximal tip element 908 and a proximal cylindrical centering element 906 (formed on the proximal side of the most proximal cleaning and/or drying element 300). Tip element 908 preferably comprises a substantially cylindrical plug-shaped element that forms the proximal end of device 910. Tip element 908 functions to guide device 910 through the straw 904 and to displace moisture and/or biomatter from the internal surface of the straw. In certain embodiments, device 910 may also include a distal tip element (not labeled) with substantially the same design as proximal tip element 908.

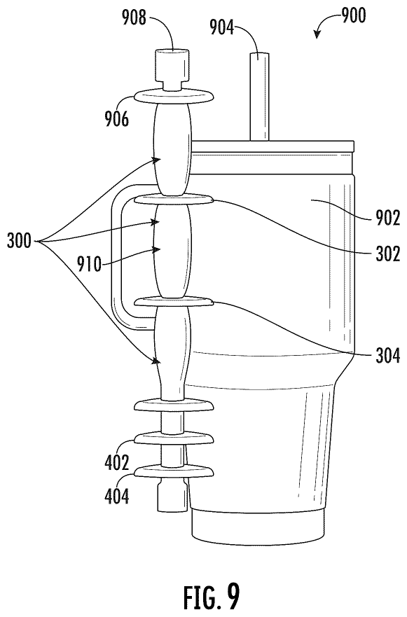

FIG. 9 illustrates a kit 900 that includes a water bottle 902 having a straw 904 and a cleaning and/or drying device 910 for the straw 904. In this embodiment, device 910 may be used to clean and/or dry the internal surfaces of drinking containers and components of drinking containers, such as drinking straws, beverage containers, water containers, bottles, thermoses, canteens, jugs, carafes, baby bottles, water bottles and insulated beverage containers and drinking straws for any of these containers.

As shown, device 910 comprises multiple cleaning and/or drying elements 300 with each cleaning and/or drying element 300 including proximal and distal end portions 302, 304 and a variable pressure region 306 therebetween, as described above. Proximal and distal end portions 302, 304 are preferably cylindrical elements having an outer diameter substantially the same as the inner diameter of the lumen to be cleaned (as discussed above). In this embodiment, the cleaning and/or drying elements are coupled to each other at the proximal and distal end portions. Cleaning and/or drying device 910 includes two additional cylindrical elements 402, 404 at a distal end of the device 910.

In this embodiment, device 910 further includes a proximal tip element 908 and a proximal cylindrical centering element 906 (formed on the proximal side of the most proximal cleaning and/or drying element 300). Tip element 908 preferably comprises a substantially cylindrical plug-shaped element that forms the proximal end of device 910. Tip element 908 functions to guide device 910 through the straw 904 and to displace moisture and/or biomatter from the internal surface of the straw. In certain embodiments, device 910 may also include a distal tip element (not labeled) with substantially the same design as proximal tip element 908.

In certain embodiments, the drying devices described above further include a programmable motor (not shown) that may be part of, or separate from, elongate member. The programmable motor is designed to withdraw the member from the internal lumen of endoscope 10 at a fixed or variable velocity. Alternatively, the motor may be programmed with a particular algorithm that corresponds to certain cleaning objectives. In one embodiment, the motor is programmed to withdraw the elongate member at a fixed velocity based on established cleaning times required to completely absorb and remove biomatter from the internal lumen. In an alternative embodiment, the motor is programmed to withdraw the elongate member in a series of discrete steps, i.e., holding the shaft in place for a specified period of time and then withdrawing it a specified distance and repeating this step until it has been withdrawn and the cleaning procedure is complete.

The drying devices described herein may include one or more sensors (not shown) along the elongate member for detecting biomatter, pathogens, liquids or other particulate matter within the endoscope 10. Suitable sensors may include PCT and microarray based sensors, optical sensors (e.g., bioluminescence and fluorescence), piezoelectric, potentiometric, amperometric, conductometric, nanosensors or the like. The devices may further include an indicator, such as a display, coupled to the sensor(s) and configured to indicator the presence of biomatter pathogens, liquids or other particulars detected by the sensor. The indicator may be any suitable chemical indicator validated for cleaning and/or sterilization procedures that undergoes a physical or chemical change visible to the human eye after exposure to certain parameters. The indicator and sensor may be part of the same device, or separate from each other.

Hereby, all issued patents, published patent applications, and non-patent publications that are mentioned in this specification are herein incorporated by reference in their entirety for all purposes, to the same extent as if each individual issued patent, published patent application, or non-patent publication were specifically and individually indicated to be incorporated by reference.

While several embodiments of the description have been shown in the drawings, it is not intended that the description be limited thereto, as it is intended that the description be as broad in scope as the art will allow and that the specification be read likewise. Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Various alternatives and modifications can be devised by those skilled in the art without departing from the description. Accordingly, the present description is intended to embrace all such alternatives, modifications and variances. As well, one skilled in the art will appreciate further features and advantages of the present description based on the above-described embodiments. Accordingly, the present description is not to be limited by what has been particularly shown and described, except as indicated by the appended claims.

What is claimed is:

1. A drying device for use with an endoscopic instrument, the device comprising:
an elongate member configured for advancement through a lumen within the endoscopic instrument;
at least one drying member coupled to a portion of the elongate member, wherein the drying element includes at least one portion that has an outer diameter greater than an outer diameter of the elongate member; and
wherein the elongate member and the drying member are sterilized or disinfected.

2. The device of claim 1, wherein at least a portion of the drying member is shaped and configured to substantially maintain contact with a wall of the lumen as the elongate member is advanced through the lumen.

3. The device of claim 1, wherein the drying member comprises one or more substantially annular members extending radially outward from the drying member.

4. The device of claim 3, wherein the annular members are discs.

5. The device of claim 3, wherein the annular members are spaced from each other along the elongate member.

6. The device of claim 3, wherein the annular members comprise a first annular member having a first diameter and a second annular member having a second diameter, wherein the first diameter is greater than the second diameter.

7. The device of claim 3, wherein the annular members are spaced from each other along the elongate member a substantially equal distance.

8. The device of claim 3, further comprising a first, second and third annular member, wherein the first and second annular members are spaced from each other by a distance greater than the second and third annular members.

9. The device of claim 1, wherein the elongate member comprises a first end and a second end, and further comprising a loop coupled to the first end.

10. The device of claim 1, further comprising a centering element coupled to either the elongate member or the drying member and configured to center the drying member within the lumen.

11. The device of claim 10, wherein the centering element has an outer dimension about 50 percent to about 90 percent of an internal diameter of the lumen.

12. A kit for cleaning and drying an endoscopic instrument, the kit comprising:
a drying device comprising an elongate member configured for advancement through a lumen within the endoscopic instrument and at least one drying member coupled to a portion of the elongate member, wherein at least a portion of the drying member has an outer diameter greater than an outer diameter of the elongate member; and
a cleaning device comprising a shaft configured for advancement through a lumen within the endoscopic instrument and at least one cleaning member coupled to a portion of the shaft.

13. The kit of claim 12, wherein the drying device is sterilized or disinfected.

14. The kit of claim 12, wherein the drying member is shaped and configured to substantially maintain contact with a wall of the lumen as the elongate member is advanced through the lumen.

15. The kit of claim 12, wherein the drying device is coupled to the cleaning device.

16. The kit of claim 12, wherein the cleaning member is shaped and configured to substantially maintain contact with a wall of the lumen as the elongate member is advanced through the lumen.

17. The kit of claim 12, wherein the cleaning member comprises distal and proximal end portions and a central portion between the distal and proximal end portions and wherein the central portion is shaped to create a pressure gradient along the central portion from the distal end portion to the proximal end portion.

18. A kit comprising:
a beverage container comprising a drinking straw; and
an elongate member configured for advancement through a lumen of the drinking straw;
at least one drying member coupled to a portion of the elongate member, wherein the drying member comprises distal and proximal end portions and a central portion between the distal and proximal end portions; and
wherein the central portion is shaped to create a pressure gradient along the central portion from the distal end portion to the proximal end portion.

19. The kit of claim 18, wherein the pressure gradient causes an increase in a relative velocity between the drying member and fluid and air within the lumen as the drying member is advanced through the lumen.

20. The kit of claim 18, wherein the pressure gradient causes an increase in shear stress between fluid and air in the lumen and an internal wall of the lumen.

21. The kit of claim 18, wherein the central portion of the drying member comprises a contraction section coupled to the proximal end portion, a diffusion section coupled to the distal end portion and a throat section coupling the diffusion and contraction sections, wherein the throat section has a diameter less than the diameter of the proximal and distal end portions and greater than a diameter of the diffusion and contraction sections.
